(19) United States
(12) Patent Application Publication
(10) Pub. No.: US 2009/0018212 A1
(43) Pub. Date: Jan. 15, 2009

(54) METHODS OF RELIEVING STRESS

(75) Inventors: Rosemarie Einstein, New South Wales (AU); Nickolas Anastasios Lavidis, Queensland (AU)

Correspondence Address: KNOBBE MARTENS OLSON & BEAR LLP, 2040 MAIN STREET, FOURTEENTH FLOOR, IRVINE, CA, 92614, US

(73) Assignee: Neuroscent Pty Ltd., Queensland (AU)

(21) Appl. No.: 12/064,811

(22) PCT Filed: Aug. 24, 2006
(86) PCT No.: PCT/AU2006/001231

§ 371 Date: Jul. 2, 2008

(30) Foreign Application Priority Data
Aug. 24, 2005 (AU) 2005904605

(51) Int. Cl. A61K 31/11

(52) U.S. Cl. 514/703

(57) ABSTRACT

Methods, combinations and compositions for relieving or preventing the effects of stress are provided. In particular, combinations and compositions comprising cis-3-hexen-1-ol, trans-2-hexenal and α-pinene, articles of manufacture impregnated with the combination or composition and methods of relieving stress by exposure to the combination or composition are described.

### FIELD OF THE INVENTION

The invention relates to methods and combinations for relieving or preventing the effects of stress, particularly the effects of chronic exposure to stress. More particularly the invention relates to methods, compositions and articles for relieving or preventing the effects of chronic exposure to stress comprising exposing a subject to a combination of scents. Compositions containing the combination of scents and articles impregnated with or including the combination of scents that are useful in the methods of the invention are also provided.

### BACKGROUND OF THE INVENTION

Stress has been defined as a state in which an animal encounters adverse conditions that induce changes in normal physiological function (Dunn et al., 1972; Fraser et al., 1975; McCarty et al., 1981; Gamallo et al., 1988). The acute stress response, also referred to as the “fight or flight” response, serves to prepare an animal to cope with a life threatening situation and is thus vital in day to day life. However, chronic exposure to stress results in many adverse effects that may deteriorate the health of an animal and may eventually become life threatening. For example, chronic exposure to stress may result in hypertension, immune-suppression, indigestion, depression, anxiety, loss of libido, headaches, loss of memory, lack of concentration, learning difficulties and insomnia (Chrousos and Gold, 1992; Cacioppo, 1994; Chrousos et al., 1995; Cacioppo et al., 1998).

Although stress is inevitable in everyone's life, at least 10% of the population in Australia have reported stress to be a mental well being problem. Of this 10% of the population, about two-thirds of adults were classified as being exposed to low level psychological stress, about one-quarter to moderate stress and about 12% to high levels of stress (Australian Bureau of Statistics, National Health Survey—Summary of Results, 25 Oct. 2002). A report from the University of Greenwich, United Kingdom, indicated that stress and its associated emotional and physical disorders cost the community about £1.3 billion per year (http://www.gre.ac.uk, “Managing Stress”). Accordingly there is a need for methods of relieving or preventing the effects of chronic exposure to stress.

The response to stress has been shown to be affected by olfaction (see review by Kelly et al., 1997). For example, bilateral bulbectomised rats show extremely high levels of glucocorticoids in the presence of stressors (O'Connor and Leonard, 1984). Olfactory bulbectomised rats show hyperactivity in their hypothalamus-pituitary-adrenal cortex axis during stress, with high plasma levels of adrenocorticotropic hormone (ACTH) and corticosterone after 15 minutes of immobilising stress (Marcilhac et al., 1999). Odours from various sources are known to affect animals. For example, rat odour has been shown to increase sympathetic neurotransmission in mice (D'Arbe et al., 2002), a combination of cis-3-hexenol and trans-3-hexenal has been reported to attenuate the acute increase in body temperature associated with a psychological stressor (Akutsu et al., 2002) and to alter the electroencephalogram (EEG) of humans (Sano et al., 2002). Alpha-pinene extracted from coniferous trees has been shown to prolong the resting period of rats as well as having a hypnotic effect on humans (Sano et al., 1998).

Advantageously, it has been found that a combination of three scents, cis-3-hexen-1-ol, trans-2-hexenal and α-pinene, provides an effective means for relieving or preventing the effects of chronic exposure to stress without affecting the “fight or flight” response which is vital for survival. Albeit to a lesser extent, it has also been found that a combination of cis-3-hexen-1-ol and trans-2-hexenal provides a means for relieving or preventing the effects of chronic exposure to stress.

### SUMMARY OF THE INVENTION

According to one aspect of the invention there is provided a combination comprising cis-3-hexen-1-ol, trans-2-hexenal and α-pinene. The combination may be provided in the form of a composition comprising cis-3-hexen-1-ol, trans-2-hexenal and α-pinene.

According to another aspect of the invention there is provided an article impregnated with or including a combination comprising cis-3-hexen-1-ol, trans-2-hexenal and α-pinene.

In some embodiments of the invention the cis-3-hexen-1-ol (CH) is present in the combination or composition in an amount of 0.003% to 0.3% volume, the trans-2-hexenal (TH) is present in an amount of 0.003% to 0.3% volume and the α-pinene (ap) is present in an amount of 0.001% to 0.15% volume. In some embodiments the ratio of CH:TH:ap in the combination or composition is in the range of 0.5-2:0.5-2:0.25-2. In some embodiments the combination or composition further comprises other components such as carriers, adjuvants, excipients, further odour components, cleaning components, hygiene components, colouring or polishing components, adhesive or coating components.

In some embodiments, the composition is a cleaning composition, a personal hygiene composition, a massage oil, a laundry detergent, a furniture polish, a shoe polish, a cosmetic product, an air freshener, a fragrance composition, an ink composition, adhesive composition or a coating composition.

In some embodiments, the article is paper, cardboard, plastic or fabric, for example, impregnated paper or cardboard used to freshen or perfume a home, office or car, cardboard or plastic strips designed to be placed in a battery operated odour-emanating device or an odour-emanating device attached to a power point, writing paper, envelopes, books, newspapers, instruction manuals, magazines, cleaning cloths, dusters, shoe polishing cloths, handkerchiefs, bedroom, bathroom or table linen or plastic or polymeric items able to release odours, particularly slow release of odours.

According to another aspect of the invention, there is provided a method of relieving or preventing in a subject the effects of chronic exposure to stress comprising exposing the subject to a combination or composition comprising cis-3-hexen-1-ol, trans-2-hexenal and α-pinene.

According to yet another aspect of the invention, there is provided a use of cis-3-hexen-1-ol, trans-2-hexenal and α-pinene in the manufacture of a composition for relieving or preventing the effects of chronic exposure to stress.

### DESCRIPTION OF THE INVENTION

### 1. Combinations, Compositions and Articles

The combinations of the main aspect of the invention comprise cis-3-hexen-1-ol (CH), trans-2-hexenal (TH) and α-pinene (ap). This combination may be used in neat form, or diluted in a carrier, as a concentrate or may be added as an additive, or each component added separately to a composition, either during manufacture of the composition or before or during use of the composition. The combination, as a concentrate or diluted in a carrier or as part of a composition, may also be used to impregnate an article.

In preferred embodiments, the ratio of CH:TH:ap in the combination is in the range of about 0.5-2:0.5-2:0.25-2. In some embodiments, the CH and TH are present in equal amounts and the ap is present in the same amount as the CH and TH or less than the amount of CH and TH. A preferred ratio of CH:TH:ap is in the range of about 1:1:0.1-1, preferably 1:1:0.3-0.7, most preferably 1:1:0.5. If relaxation is desired, a suitable ratio is 1:1:0.3-1, for example, 1:1:0.5 or 1:1:0.8. In other embodiments, the ap may be present in an amount the same as or greater than the CH and TH, for example 1:1:1-2. If relaxed sleep is desired, more ap may be used, for example, 1:1:1-2, such as 1:1:1.5 or 1:1:2. If a delay in the onset of hypertension caused by chronic stress, an improvement of memory or learning which has been detrimentally affected by chronic stress or relieving, preventing or delaying depression caused by chronic stress is required, the preferred ratio of CH:TH:ap is in the range of 1:1:0.3-0.5.

In preferred embodiments, the CH is present in an amount of about 0.003% to about 0.3%, especially about 0.004% to about 0.2%, about 0.005% to about 0.1%, about 0.01% to about 0.05%, more especially about 0.03% by volume of the composition. In preferred embodiments, the TH is present in an amount of about 0.003% to about 0.3%, especially about 0.004% to about 0.2%, about 0.005% to about 0.1%, about 0.01% to about 0.05%, more especially about 0.3% by volume of the composition. In preferred embodiments, the ap is present from about 0.001 to about 0.6%, especially about 0.001% to about 0.15%, especially about 0.003% to about 0.13%, 0.005% to about 0.1%, about 0.01% to about 0.05%, more especially about 0.01% to about 0.02%, especially about 0.015% by volume of the composition.

In its simplest form the three components may be mixed together in an appropriate ratio to form the combination of the invention or may be mixed together in a carrier solution or solvent. Alternatively, the combination may be included in a composition which may further comprise other components such as carriers, adjuvants, excipients, further odour producing compounds such as perfumes, cleaning components such as soaps and detergents, colouring agents such as dyes and pigments or polishing components.

The components of the combination of the invention may be formulated alone or together with a carrier or solvent and added to another liquid, semi-solid or solid composition. Alternatively the components of the combination of the invention may be added separately to a liquid, semi-solid or solid composition. Suitable liquid, semi-solid or solid compositions include, but are not limited to, cleaning products e.g.: bathroom cleaners, kitchen cleaners, floor cleaners, carpet cleaners, polishing fluids, gels, and pastes, solid or liquid soaps, shampoos, conditioners, shaving soaps and foams, bubble bath, solid, liquid or gelled laundry detergents, shoe polishes, cosmetics and other products such as massage oils, perfumes, air fresheners, burner oils, candles, inks and paints. Since the scents of the invention are sensitive to heat, care must be taken if they are to be presented in a form in which they will be exposed to heat, such as a candle or oil burner.

In another aspect of the invention, the components of the combination may be formulated together alone or with a carrier or solvent and impregnated into a material. For example, the components may be dissolved in a liquid carrier or solvent and allowed to soak into a material. Suitable materials include paper, cardboard, plastic, wood, plant materials or fabric. Suitable liquid carriers include aqueous solvents such as water or aqueous alcohols and volatile solvents that will rapidly evaporate to leave the material dry and impregnated with the composition. Suitable impregnated materials include, but are not limited to, cardboard, paper, plastic or polymeric strips that are placed or suspended in an enclosed space such as a car, room or office, optionally in front of a fan or air conditioning vent, strips that are inserted in devices attached to a power point allowing slow release into a room, cleaning and polishing cloths, candles, writing pads, paper, envelopes, books, magazines, newspapers, instruction booklets or manuals, household linen and pot pouri. The odours may also be impregnated into plastic or polymeric materials designed to release the odours slowly. Articles containing writing, for example books, magazines, newspapers, instruction booklets or instruction manuals may be impregnated during printing using an ink composition comprising the combination of the invention.

In another aspect of the invention, the components of the combination are formulated into a concentrate which is suitable for addition to another liquid, semi-solid or solid composition or for use impregnating materials. Such a concentrate may be a neat mixture of CH, TH and ap or may include a carrier or solvent. Suitable carriers or solvents include water, triethyl citrate, alcohols such as methanol or ethanol. In some cases, hydrocarbon carriers may be used, for example, pentane or hexane. Preferably the ratio of CH:TH:ap in the concentrate is in the range of about 0.5-2:0.5-2:0.25-2. In some concentrates the CH and TH are present in equal amounts and the ap is present in the same amount as CH and TH or less than the amount of CH and TH. In an especially preferred concentrate, the CH:TH:ap ratio is 1:1:0.5. In other concentrates, the CH and TH are present in equal amounts and the ap is present in the same amount as CH and TH or more than the amount of CH and TH. In a preferred embodiment, a concentrate may have a CH:TH:ap ratio of 1:1:2.

Although the combination provides a distinct odour that can be used as a fragrance in a composition or article of the invention, it is not necessary that the combination be present in the composition or article at a concentration that can be detected by the subject. Therefore, in some embodiments, the odour of the combination may be masked by other perfumes or fragrances. Non limiting examples of other fragrances include essential oils such as bergamot, cedarwood, sage, chamomile, cypress, eucalyptus, frankincense, geranium, grapefruit, juniper berry, lavender, lemon, sweet marjoram, myrrh, sweet orange, patchouli, peppermint, petitgrain, rosemary, rosewood, sandalwood, tea tree and ylang ylang and raw perfume materials, such as Dodecahydro-3a,6,6,9a-tetramethylnaphtho[2,1-b]furan, dodecahydrotetramethylnaphthofuran, 1-(2,2,6-trimethylcyclohexyl)-3-hexanol, oxacycloheptadec-10-en-2-one, trimethyl-bicyclo-heptane-spiro-cyclohexenone, 4-(2,2,6-trimethyl-1-cyclohexen-1-yl)-3-buten2-one, decahydro-2,6,6,7,8,8-hexamethyl-8H-Indenol(4,5-B)furan, 4-(2,6,6-trimethyl-1-cyclohexen-2-yl)-3-buten-2-one, 5-(2,6,6-trimethyl-1-cyclohexen-1-yl)-4-penten-3-one, octahydro-2,3,8,8-tetramethyl-2-acetonaphthone, methyl 2,4-dihydroxy-3,6-dimethylbenzoate, 3,3-dimethyl-5-(2,2,3-trimethyl-3-cyclopenten-1-yl)-4-penten-2-ol, 3-methyl-5-(2,2,3-trimethyl-3-cyclopenten-1-yl)-4-penten-2-ol, 2-ethyl-4-(2,2,3-trimethylcyclopent-3-enyl)-2-buten-1-ol, 1-(2,6,6-trimethyl-3-cyclohexen-1-yl)-2-buten-1-one, 4-(2,6,6-trimethyl-2-cyclohexenyl)-2-buten-4-one, 4-(2,6,6-trimethylcyclohex-1-enyl)-2-buten-4-one, 3-methoxy-4-hydroxybenzaldehyde, 3-ethoxy-4-hydroxybenzaldehyde, 1,4-decanolide, 1,5-decanolide, 4-n-amyl-4-hydroxybutyric acid lactone, 1,4-dodecanolide, 1,5-dodecanolide, 4-n-heptyl-4-hydroxybutanoic acid lactone, 5-n-hexyl-5-hydroxypentanoic acid lactone, 4-(2,6,6-trimethylcyclohexa-1,3-dienyl)but-2-en-4-one, (Z)-4-cyclopentadecen-1-one, tetrahydro-6-(3-pentenyl)-2H-pyran-2-one, (Z)-5-(3-hexenyl)dihydro-5-methyl-2(3H)-furanone, 5-methyl-5-hexyl-tetrahydrofuran-2-one, 4-(4-hydroxyphenyl)-2-butanone, ethyl 3-methyl-3-phenyloxiranecarboxylate, 2-ethyl-3-hydroxy(4H)pyran-4-one, 1,3-benzodioxole-5-carboxaldehyde, adoxal, allyl amyl glycolate, allyl cyclohexane propionate, alpha terpineol, amyl cinnamic aldehyde, amyl salicylate, anethol, anisic aldehyde, benzaldehyde, benzyl acetate, benzyl acetone, benzyl alcohol, benzyl benzoate, benzyl salicylate, beta gama hexenol, beta naphthol methyl ether, beta pinene, borneol, bornyl acetate, bourgeonal, butyl anthranilate, calone, camphor, carvacrol, carvone, cahmeran, cis-3-hexenyl acetate, cis-3-hexenyl benzoate, cis-3-hexenyl salicylate, cis-1,3-oxathiane-2-methyl-4-propyl acetate, citral, citronellal nitrile, citronellol, citronellyl acetate, coronol, coumarin, 8-cyclohexadecen-1-one, cyclogalbante, cyclal C, cyclemax, cymal, decyl alcohol, decyl aldehyde, delta muscenone, dihydro iso jasmonate, dihydro myrcenol, dimethyl benzyl carbinol, dimetol, dimethyl benzyl carbinyl butyrate, dimethyl benzyl carbinyl isobutyrate, ethyl acetoacetate, ethyl butyrate, ethyl caproate, thelyene brassylate, ethyl-2-methyl butyrate, ethyl linalool, eucalyptol, eugenol, flor acetate, florol, florosa, exaltolide/cyclopentadecanolide, floralozone, florhydral, frutene, fructone, galaxolide, geraniol, geranyl acetate, geranyl nitrile, habanolide, helional, herbavert, hexyl acetate, hexyl cinnamic aldehyde, hexyl salicylate, hivernal, hydroquinone dimethyl ether, hydroxycitronellal, indol, indolene, intreleven aldehyde, ionone gamma methyl, iso-amyl acetate, iso-amyl alcohol, isocyclo citral, isoeugenol, isoeugenol acetate, isopropylquinoline, lilial, limonene, linalool, linalool oxide, linalyl acetate, lyral, mandarinal, menthol, melonal, 2-methoxy-3-(2-methylpropyl)-pyrazine, methyl anthranilate, methyl beta naphthyl ketone, methyl cedrylone, methyl dihydro jasmonate, methyl heptine carbonate, methyl isobutenyl tetrahydropyran, methyl octine carbonate, methyl phenyl carbinyl acetate, myrecene, neobutenone, neofolione, nerol, 2,6-nonenol, phenoxy ethyl isobutyrate, phenyl acetaldehyde dimethyl acetal, phenyl carbinyl acetate, phenyl ethyl dimethyl carbinol, phenyl propyl alcohol, prenyl acetate, 2,6-nonadienal, 2,6-nonadien-1-ol, nonyl aldehyde, octyl aldehyde para cymene, para cresol, pentalide, phenyl acetaldehyde, phenoxanol, phenoxy ethyl propionate, sandalore, sandela, tetrahydrolinalool, thymol, tridecene-2-nitrile, trifone, triethyl citrate, triplal, tuberosa, undecavertol, undecylenic aldehyde, undecyl aldehyde, veloutone, verdox, vernaldehyde, zingerone and combinations thereof.

Suitable compositions containing the combination of the invention are as follows:

### a) Cleaning Composition

In one embodiment of the invention the combination comprising CH, TH and ap is incorporated in a cleaning composition and further comprises components suitable for cleaning products. Optional suitable components include, but are not limited to, surfactants, polymers, organic solvents, polycarboxylic acids, peroxides, thickening agents, aqueous solvents, suds suppressors, perfumes, detergency builders, buffers, preservatives, antibacterial agents, colorants, bleaching agents, chelators, enzymes, hydrotropes, propellants, corrosion inhibitors and mixtures thereof. Such compositions may be prepared by methods known in the art by incorporating the combination of the invention with perfumes or fragrances being incorporated in the composition or by substituting the combination of the invention for perfumes or fragrances normally used in the composition. A person skilled in the art of cleaning composition formulation can readily prepare a cleaning formulation comprising a combination of CH, TH and ap according to the present invention and determine suitable components based on the purpose of the cleaning composition.

Suitable cleaning compositions include household cleaners such as bathroom, toilet and shower cleaners and kitchen cleaners, hard surface cleaners such as floor cleaners, soft surface cleaners such as carpet cleaners or laundry detergents. The cleaning compositions may be in liquid, semi-solid or gel form, or solid form (such as granulates). Such cleaning compositions provide exposure to the combination of the invention while cleaning and may result in the slow release of the combination of CH, TH and ap containing composition thereby exposing humans or animals in the vicinity of the cleaning composition or the cleaned surface to the chronic stress relieving effects of the composition.

### b) Personal Hygiene Products and Cosmetic Compositions

The combination comprising CH, TH and ap or each component separately may be incorporated into personal hygiene products and cosmetics in a similar manner to the incorporation of other fragrance components in such compositions, as known in the art.

Suitable personal hygiene and cosmetic products include, but are not limited to, bar soaps, liquid soaps, body washes, shampoos, conditioners, hair mousse, gel or other products, bubble bath, shaving gels, soaps or foams, facial and body creams, lotions and fragrance products. Such products may comprise in addition to the combination of CH, TH and ap, other components such as surfactants, emollients, thickeners, moisturisers, alcohols, other fragrances, dyes, anti-dandruff agents, vitamins, chelating agents, humectants, pH adjustors, extracts, pearlants, UV absorbers, anti-oxidants, oils, buffers and propellants. Those skilled in the art could prepare such compositions by known methods and also determine suitable components based on the purpose of the personal hygiene product or cosmetic product.

Not only are those using the personal hygiene and cosmetic products exposed to the combination comprising CH, TH and ap during use, but residual composition remaining on the skin or hair may provide continued exposure to the combination after use of the personal hygiene or cosmetic product.

### c) Polish Compositions

The combination comprising CH, TH and ap or each component separately may be incorporated into polishing compositions, such as furniture polish, floor polish or shoe polish, in a similar manner to incorporation of other fragrances in such compositions.

Such polishes may, depending on purpose, include components such as waxes and oils, water proofing components, such as silicone, and dyes. Suitable waxes include, but are not limited to, paraffin wax, microcrystalline wax, bees wax or mixtures thereof. Other optional components include surfactants, anti-oxidants, e.g. sodium benzoate, preservatives, e.g. chloromethylisothiazolinone or benzoisothiazoline-one, anti-static agents, perfumes, wood oil extracts and propellants.

Not only are those using the polish compositions exposed to the combination of CH, TH and ap while polishing furniture, shoes or other surfaces such as floors, but residual odour may be slowly released from the furniture, shoes, or surfaces exposing humans and animals in their vicinity to the chronic stress relieving effects of the combination of the invention.

### d) Massage Oils

The combination comprising CH, TH and ap or each component separately, may be incorporated into a massage oil. The combination, as a concentrate or as a diluted solution, may be combined with the oil directly before use or may be incorporated in the massage oil at the time of manufacture.

Suitable components of massage oils include liquid oils or semi-solid oils that form liquids at body temperature, including, but not limited to, sesame seed oil, almond oil, canola oil, olive oil, grapeseed oil, jojoba oil, peanut oil, safflower oil, kukui nut oil, castor oil, borage oil, avocado oil, apricot kernel oil and wheat germ oil; essential oils, such as bergamot, cedarwood, sage, chamomile, cypress, eucalyptus, frankincense, geranium, grapefruit, juniper berry, lavender, lemon, sweet marjoram, myrrh, sweet orange, patchouli, peppermint, petitgrain, rosemary, rosewood, sandalwood, tea tree and ylang ylang; and vitamins, such as vitamin E.

Such massage oils provide exposure of both the subject being massaged and the masseuse to the combination comprising CH, TH and ap during the massage and while the subject is relaxed or becoming relaxed. Furthermore, any massage oil composition remaining on the skin after the massage is finished will provide ongoing exposure to the combination of CH, TH and ap.

### e) Air Fresheners

The combination comprising CH, TH and ap may be incorporated into solid or liquid air fresheners or in air freshening or deodorising strips that fit in battery operated odour releasing devices or devices designed to fit a power point and release odour while the power is supplied. The combination of the invention may be incorporated by known methods with addition of the combination to or with perfumes or fragrances being added to the air freshening composition, or may replace or be substituted for perfumes or fragrances normally used in the air freshener. The combination comprising CH, TH and ap may be absorbed into an absorbent material, for example, by immersing the absorbent material into the combination or a composition containing the combination. The absorbent material impregnated with the combination is then placed in a device or in a position that allows evaporation providing release of the combination into the environment.

### f) Inks

The combination comprising CH, TH and ap or each component separately may be incorporated into an ink composition and used for printing on paper, either manually or by means of typewriter or computer printers, such as inkjet printers. Further suitable components for inks include pigments and dyes and carriers for the pigments, dyes and combination of the invention. Such inks are described, for example, in U.S. Pat. No. 6,656,256 incorporated herein by reference.

Such ink compositions not only provide exposure of the printing workers to the combination of the invention but also those readers of the printed product within a period of time after printing.

### g) Paints

The combination comprising CH, TH and ap may be formulated as an additive to scent paint or each component may be added to a pre-formed paint composition separately. A suitable additive composition for adding to a pre-formed paint composition includes CH, TH and ap and optionally emulsifiers such as non-ionic emulsifiers or blends of non-ionic and anionic emulsifiers; and solubilising agents such as diethyl phthalate, dimethyl phthalate, dioctyl adipate, hexylene glycol, dipropylene glycol, 1,3-butanediol, 1,4-butanediol, kerosene, linonene, triethylacetate, dipropylene glycol, ethyl alcohol, benzyl benzoate and mixtures thereof. Other optional components include anti-bacterial/anti-microbial agents, mildewcidal agents, colouring agents, glitters and mixtures thereof. Such additive compositions are described in U.S. Pat. No. 6,838,492 incorporated herein by reference.

The components of the combination, CH, TH and ap, may also be included in the paint composition during manufacture by mixing the combination or each component separately with a paint pigment and a paint solvent, such as described in U.S. Pat. No. 5,078,792 incorporated herein by reference.

Suitable paints include, but are not limited to, water based paints, latex based paints, oil based paints, alkyd based paints and mixtures thereof.

The paint may then emit the odour of the combination while in a liquid state, before painting, during painting or during drying, and in some cases may emit odour slowly over a long period of time. In preferred embodiments, the paint is used to paint a surface in an enclosed space such as a room, office or car.

The painter and those in the vicinity of the painted surface during painting, drying of the paint and in some cases over an extended period of time after the paint has dried, will be exposed to the chronic stress relieving effects of the combination of the invention.

### h) Candles and Oil Based Fragrance Burners

The combination comprising CH, TH and ap or each component separately may be blended with wax such as paraffin wax or bees wax and moulded into candles that release the odour of the combination over a period of time, particularly when the candle is being burnt.

Similarly, the combination comprising CH, TH and ap or each component separately may be blended with oil and placed in the receptacle of a candle burner. Particularly when heat from a candle is applied, the odour of the combination of the invention is released into its surroundings exposing those subjects in its vicinity to the chronic stress relieving effects of the composition.

### i) Impregnated Paper and Cardboard Products

Cardboard and paper may be impregnated with a combination containing CH, TH and ap or a composition containing CH, TH or ap. Such products include paper including writing paper, envelopes, books, newspapers, magazines, instruction booklets or manuals. Other products may include disposable pieces of cardboard or paper, optionally cut into aesthetically pleasing shapes and optionally adorned with colours, patterns or pictures that are impregnated with the combination of the invention and placed or suspended in an enclosed space such as a room, office or car. Optionally such disposable pieces of impregnated cardboard or paper may be suspended in front of a fan or air conditioning system to circulate the odour of the combination of the invention.

Such impregnated paper and cardboard products may be prepared by coating or spraying the paper or cardboard with a combination comprising CH, TH, ap and optionally a solvent or carrier, or by immersing the paper or cardboard in a combination comprising CH, TH, ap and optionally a solvent or carrier. Suitable solvents or carriers include any solvent or carrier in which the CH, TH and ap are soluble and which does not harm the paper or cardboard. Examples of suitable solvents or carriers include water, triethyl citrate, methanol and ethanol. Particularly preferred solvents or carriers are those that readily evaporate leaving a dry and intact cardboard or paper product. Alternatively, the paper or cardboard product may be impregnated with a combination of the invention by printing or dying the paper or cardboard with an ink composition comprising the combination of the invention.

### j) Impregnated Cloth or Fabric

Cloth or fabric may be impregnated with a combination comprising CH, TH and ap by spraying, coating or immersion as described for paper products above, or by washing the fabric using a laundry detergent containing the combination of the invention.

Multi-layer fabrics may also include an impregnated layer fused to other fabric layers allowing the slow release of the combination of the invention from the fabric.

Impregnated fabric or cloth including synthetic textiles, may be used in cleaning cloths, dusters, sponges, towels, sheets, pillow cases, duvet covers or quilt covers, table cloths, napkins, tea towels, handkerchiefs, clothing and the like.

### k) Impregnated Organic Materials

Organic materials, such as wood or plant material (eg pot pouri), may be impregnated with a combination comprising CH, TH and ap by spraying, coating or immersion as described for paper products above, or by adding drops of concentrate or diluted solution of the combination of the invention to the organic material and letting it be absorbed.

### l) Impregnated Plastics or Polymers

Plastics and polymeric materials may be impregnated with a combination comprising CH, TH and ap by methods known in the art. Suitable products containing plastic or polymeric materials include for example, plastic or polymeric films used in disposable nappies, sanitary pads and incontinence pads.

### 2. Methods of Relieving or Preventing Chronic Stress

In one aspect of the present invention, there is provided a method of relieving or preventing the effects of chronic exposure to stress comprising exposing a subject to a combination comprising cis-3-hexen-1-ol, trans-2-hexenal and α-pinene.

As used herein, the term “subject” refers to air breathing animals that have a sense of smell, for example mammals, birds and reptiles. Preferred subjects are those that may be exposed to chronic stress, for example, humans, domestic and agricultural animals, animals kept in captivity and laboratory animals. Especially preferred subjects are humans and laboratory animals.

As used herein, the term “exposing a subject” refers to providing the subject or the environment in which the subject is located with the combination comprising CH, TH and ap such that the subject is able to inhale the components of the combination. In particular, the subject is able to inhale the combination through their nose allowing contact between the components of the combination and the subject's olfactory sensors. Any means of providing contact between the subject's olfactory sensors and the components of the composition may be used. For example, the combination may be released into the air in the vicinity of the subject from a composition comprising the combination or from a material impregnated or coated with the combination.

As used herein, the term “chronic exposure to stress” refers to long term exposure to stress resulting from an environment, life style or chronic situation in that is constantly emotionally and/or physically stressful. Examples of environments, life styles or chronic situations that result in chronic exposure to stress include long term serious illness of self or significant other, strict or neglectful childhood environments, dysfunctional relationships or family environments, high pressure working environments and financial hardship. Common stressors in laboratory animals that result in chronic exposure to stress include unfamiliar procedures, immobilisation, excessive noise, bright light, transportation, overcrowding, unfamiliar cage-mates, maternal-infant separation, peer isolation, handling and injections. An initial application of a stressor may provide an initial response that includes an increase in cortisol or corticosteroids, ACTH and/or catacholamines. However, as the stressor is applied over a period of time or is applied repeatedly, the stress becomes chronic.

As used herein, the term “acute stress” refers to the result of exposure of a subject to a single, or possibly multiple, traumatic events. The response to acute stress is an extremely important protective response and may be referred to as the “fight or flight” response. When subject to acute stress, the body releases adrenaline which in turn increases sugar release from the liver and increases heart rate increasing blood flow and oxygen delivery to muscles. Cortisol is also released to assist in maintaining high blood sugar. At the same time, non-essential body functions, such as digestion, shut down or diminish. The response to acute stress is essential to humans and animals allowing a rapid response to threat or danger.

The method of the present invention allows attenuation of the effects of exposure to chronic stress but does not adversely affect the body's ability to undergo a “fight or flight” response when faced with threat or danger.

Chronic exposure to stress may result in many adverse effects which affect the health of a subject and may become life threatening. Adverse effects related to chronic stress include hypertension, immune suppression, depression, anxiety, loss of libido, indigestion, head aches, loss of memory, lack of concentration, learning difficulties and insomnia.

By “relieving or preventing” is meant that the adverse effects of chronic stress are reduced or to at least some extent removed or the onset of the adverse effect(s) is delayed or halted or partially or fully reversed.

In some embodiments, the method of the invention delays the onset of or prevents hypertension that is caused by chronic stress. In other embodiments, the method of the invention may improve concentration, memory and learning in chronically stressed subjects.

In some embodiments, the methods of the invention can be used as an adjunct therapy together with other therapy for treating the adverse effect of chronic stress. For example, the methods of the invention may be used together with another therapy, for example a conventional therapy, for hypertension, immune suppression, depression, anxiety, loss of libido, indigestion, head ache, loss of memory, lack of concentration, learning difficulties or insomnia.

In yet other embodiments, the methods of the invention may be used to delay the onset of or reduce the severity of post-traumatic stress disorder, particularly in persons whose occupations expose them to high levels of stress, continually or periodically. Such occupations include, but are not limited to, police, military, prison staff and inmates, civil or medical emergency staff.

In still other embodiments, the methods of the invention may be used as an adjunct therapy in psychotherapy. For example, patients having psychotherapy for mental disorders such as depression, anxiety and post-traumatic stress syndrome, may be exposed to the combination of the invention during psychotherapy sessions.

In a common embodiment, the methods of the invention are used for relaxation and to relieve tension caused by chronic stress.

As used herein, the term “effective amount” relates to an amount of the combination of the invention, which when exposed to a subject, provides the desired relief or prevention of the effects of chronic exposure to stress. Exposure of the subject to the combination of the invention may occur at intervals of minutes, hours, days, weeks, months or years, or may occur continuously. A relieving or preventing effective amount of composition is an amount, which upon exposure of the subject at least partially attains the desired therapeutic effect or delays the onset of, inhibits the progression of or halts or partially or fully reverses the onset or progression of the adverse effects caused by chronic stress.

In some embodiments, stress relief may be achieved with a composition of the invention having 0.03% vol CH:0.03% vol TH:0.015% vol ap where 0.5 to 1 mL is released into a room of about 80 cubic meters (7 m×8 m×4 m) over 6 hours. A person of skill in the art could readily determine a suitable amount of combination and a suitable ratio of components to provide the level of stress relief required in a particular environment.

Although the combination provides a distinct odour that can be used as a fragrance in a composition or article of the invention, it is not necessary that the combination be present in the composition or article at a concentration that can be detected by the subject. The odour of the combination of the invention is still capable of relieving or preventing the effects of chronic exposure to stress even if its odour is not detectable by the subject or if the odour is masked by other perfumes. It is therefore possible to provide a method of relieving or preventing the effects of chronic exposure to stress with a composition or impregnated article that does not have a green odour associated with cis-3-hexen-1-ol and trans-2-hexenal or the pine odour of α-pinene.

In another aspect of the method of the invention, the inclusion of α-pinene in the combination used is optional. According to this aspect, there is provided a method of relieving or preventing the effects of chronic exposure to stress comprising exposing a subject to a combination of cis-3-hexen-1-ol and trans-2-hexenal. In preferred embodiments the ratio of CH:TH is 0.5-1.5:0.5-1.5, preferably 1:1. In other embodiments, the combination also comprises α-pinene.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### BRIEF DESCRIPTION OF DRAWINGS

FIG. 1 is a graphical representation showing the comparison of the effects of different odours on the change in sympathetic neurotransmitter release following chronic exposure to the stress of concomitant social deprivation and predator exposure.

FIG. 2 is a graphical representation showing stress induced increase in sympathetic neurotransmission.

FIG. 3 is a graphical representation showing the effect of a combination comprising 0.03% vol cis-3-hexen-1-ol, 0.03% vol trans-2-hexenal and 0.015% vol α-pinene on the stress induced increase in sympathetic neurotransmission.

FIG. 4 is a graphical representation of the effect of a combination comprising 0.03% vol cis-3-hexen-1-ol, 0.03% vol trans-2-hexenal and 0.015% vol α-pinene on heart rate response to 1 hour of restraint on the first day of treatment.

FIG. 5 is a graphical representation of the effect of a combination comprising 0.03% vol cis-3-hexen-1-ol, 0.03% vol trans-2-hexenal and 0.015% vol α-pinene on heart rate response to 1 hour of restraint on the 7^{th} day of treatment.

FIG. 6 is a graphical representation showing dose response curves to noradrenaline in vasa differentia from mice which had been restrained daily for 1 hour, for 14 days. Animals had been housed in the presence or absence of a combination comprising 0.03% vol cis-3-hexen-1-ol, 0.03% vol trans-2-hexenal and 0.015% vol α-pinene.

FIG. 7 is a graphical representation demonstrating the effect of different concentrations and ratios of CH, TH and ap on sympathetic neurotransmitter release as a marker of stress.

FIG. 8 is a graphical representation of the effect of 1% CH, 1% TH and 1% ap (all % by volume) on smooth muscle cells and their ability to successfully undergo neurotransmission upon exposure to stimuli.

FIG. 9 is a graphical representation of the effect of CH:TH:ap, (0.03%:0.03% 0.015% by volume) on the normalised plasma corticosterone level in non-stressed (control, I, n=2), stressed with formulation (II, n=3) and stressed without formulation (III, n=4) mice, where stress was applied for 14 days.

Those skilled in the art will appreciate that the invention described herein is susceptible to variations and modifications other than those specifically described. It is to be understood that the invention includes all such variations and modifications which fall within the spirit and scope.

The invention will now be described with reference to the following examples which are included for the purpose of illustration only and are not intended to limit the generality of the invention hereinbefore described.

### EXAMPLES

### Methods

The effects of odours on experimental animals exposed to stressors that cause chronic stress was assessed. Mice were divided into two groups, a control group exposed to minimal stress and a group exposed to severe stress by concurrent isolation and exposure to a predator. Each group was divided into sub-groups, one not exposed to any odour (control), one exposed to triethyl citrate vehicle (control), and the other groups exposed to an odour or a mixture of odours selected from cis-2-hexen-1-ol, trans-3-hexen-1-ol, trans-2-hexen-1-ol, cis-3-hexen-1-ol, trans-2-hexenal and α-pinene. Each odour compound was diluted (0.01 to 0.05% volume) with triethyl citrate. The solutions (0.5 mL) were placed on filter paper strips and placed within 30 cm of the animal cage. The response to stress was assessed using any or a combination of telemetry measurements, post-mortem electrophysiological studies and biochemical assays of blood samples.

### Telemetry Transmitters

Telemetry transmitters were used to record electrocardiogram (ECG) from which heart rate (HR) was derived, body temperature (BT) and locomotor activity (LA). Mice were surgically implanted with a transmitter and allowed to recover for 10 days. Previous studies have shown that parameters measured by the implanted transmitter reach a steady state and diurnal rhythm returns to normal after 10 days recovery period. This suggests complete recovery from the surgical procedure (Einstein et al., 2000; Einstein et al., 2004).

The experimental animals were then exposed to one of the stressors, with or without odour(s). Continuous telemetry monitoring HR, BT and LA was performed during the post-operative recovery period and throughout the experimental period.

### Electrophysiological Examination

Isolated vasa differentia were used to assess the effects of prolonged exposure to stress and its attenuation by odours. Following exposure of the mice to one of the stressors with or without odour, the level of neurotransmission from sympathetic varicosities was examined using standard focal extracellular recording methods (Lavidis and Bennett, 1992, 1993a, 1993b; D'Arbe et al., 2002). Following prolonged stress exposure, the level of sympathetic neurotransmission significantly increases. The extent of the increase reflects the level of stress the animal was exposed to during the previous 6-14 days (D'Arbe et al., 1999, D'Arbe et al., 2002).

### Example 1

Mice were exposed to two stressors concurrently, social deprivation and the presence of predators (rats in separate cages) for 10 to 12 days with or without the following odours or combination of odours, cis-2-hexen-1-ol, trans-2-hexen-1-ol, trans-3-hexen-1-ol, trans-2-hexenal, cis-3-hexen-1-ol, α-pinene and a combination of cis-3-hexen-1-ol and trans-2-hexenal. Two controls were used: mice not exposed to stress and mice exposed to stress together with triethyl citrate vehicle. The mice were monitored for up to 4 days after the stressors were removed.

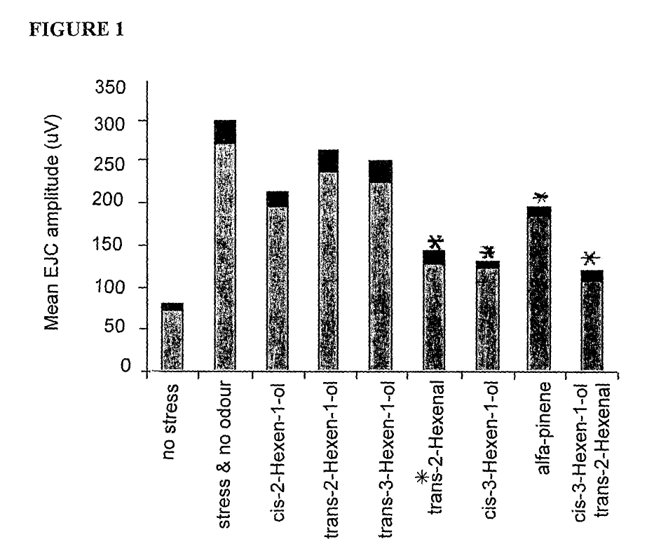

FIG. 1 shows the mean excitatory junction potentials from 12 to 15 different electrode recording sites from 3 animals for each treatment (light bars) determined by electrophysiological examination as described above. The standard deviation is shown by the dark bars with a* indicating p<0.05 and *p<0.1. The effectiveness of the odour or odour combination from most effective to least effective are combined cis-3-hexen-1-ol and trans-2-hexenal>cis-3-hexen-1-ol>trans-2-hexenal>α-pinene>cis-2-hexen-1-ol>trans-3-hexen-1-ol>trans-2-hexen-1-ol.

### Example 2

Using the method of Example 1, four combinations of odour compounds were investigated:
(i) 0.005% volume cis-3-hexen-1-ol (CH), 0.005% volume trans-2-hexenal (TH), 0.005% volume α-pinene (ap);
(ii) 0.1% volume CH, 0.1% volume TH and 0.1% volume ap;
(iii) 0.05% volume CH, 0.05% volume TH and 0.05% volume ap; and
(iv) 0.03% volume CH, 0.03% volume TH and 0.015% volume ap.

All four combinations were effective at reducing changes in sympathetic neurotransmission with the greatest reduction produced by the combination of 0.03% volume CH, 0.03% volume TH and 0.015% volume ap.

### Example 3

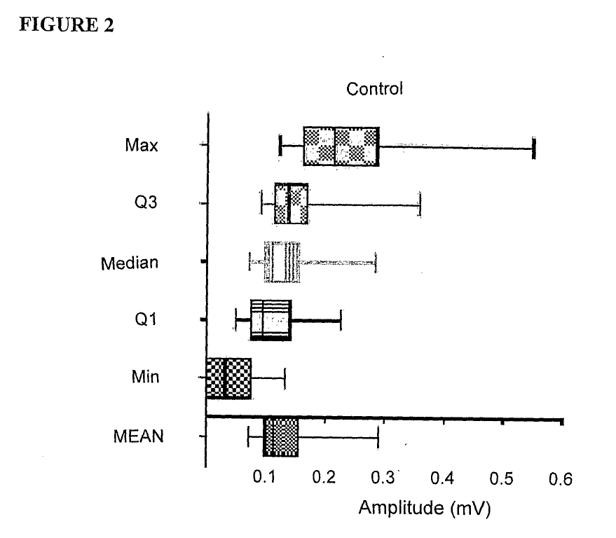

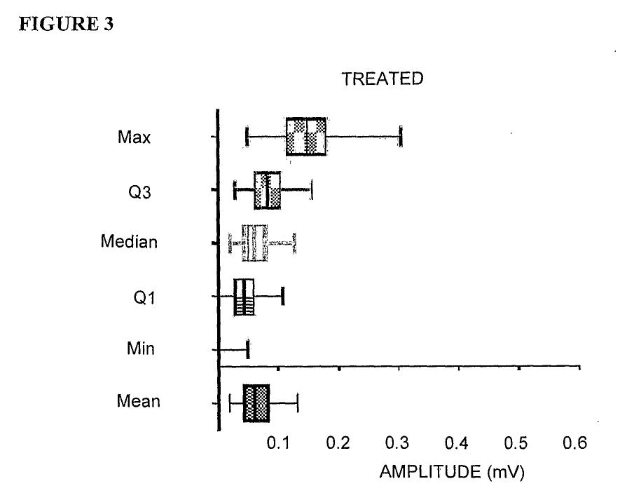

Mice were placed in cages with one mouse per cage (social deprivation) in a room that contained cages with rats (predators) without odours (control). Another group of mice were exposed to the same stress as the control but were also exposed to 0.03% volume CH, 0.03% volume TH and 0.015% volume ap (treated). Box plots of the excitatory junction currents (EJC) obtained by electrophysiological methods are shown in FIG. 2 (control) and FIG. 3 (treated) and indicate the maximum EJC amplitude (Max), the distribution of 3^{rd} quartiles (Q3), the median, the distribution of the 1^{st} quartiles (Q1) and the distribution of the means. The data is from 16 animals from each treatment with at least 40 recording sites. The EJC amplitudes are significantly reduced in the animals exposed to odours during the stress exposure (10 to 14 days).

### Example 4

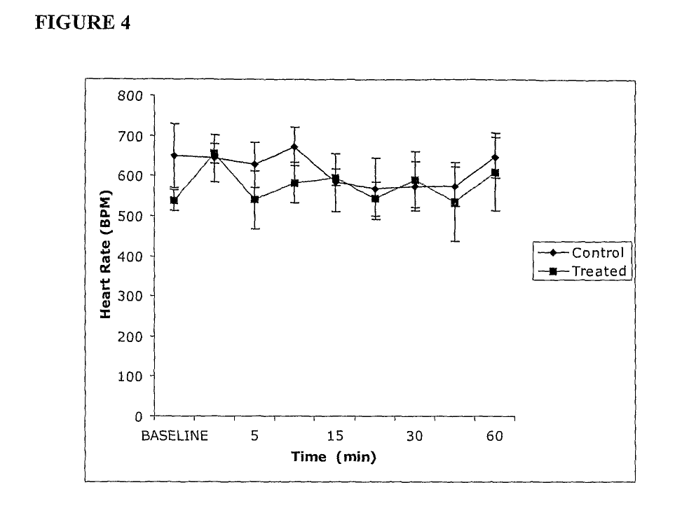

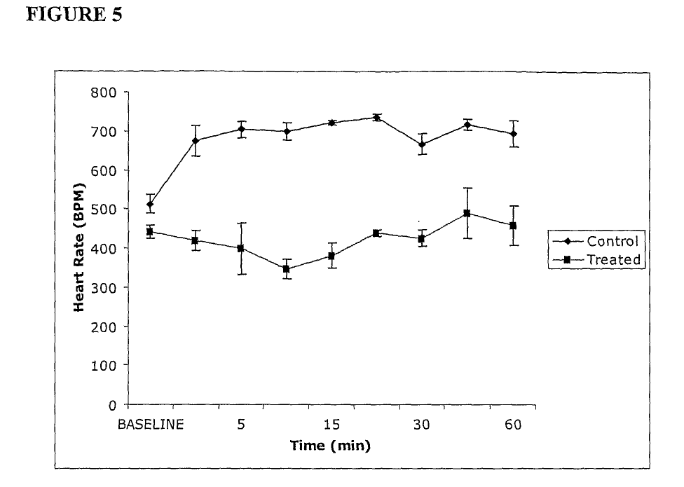

Mice were implanted with telemetric transmitters and left to recover before starting experiment. HR, BT and LA were monitored over the recovery period. Once the animals had recovered, they were subjected to 1 hour of restraint each day. In animals that had not been exposed to odours HR increased from 500 to 700 beats per minute and BT increased by 10 when the animals were restrained for 1 hour. The rise in HR and BT were abolished in mice which had been housed with odour chemicals 0.03% volume CH, 0.03% volume TH and 0.015% volume ap (in the cage). FIG. 4 shows the HR during restraint of control animals and those housed with odours on the 1^{st} day of restraint. FIG. 5 shows the HR during restraint of control animals and those housed with odours on the 7^{th} day of restraint. On the first day during the first period of restraint, the odour did not have a significant effect on heart rate compared to control animals. However, after 7 days of being subjected to restraint on a daily basis, the animals housed with odours were less responsive to the restraining stress in terms of heart rate.

### Example 5

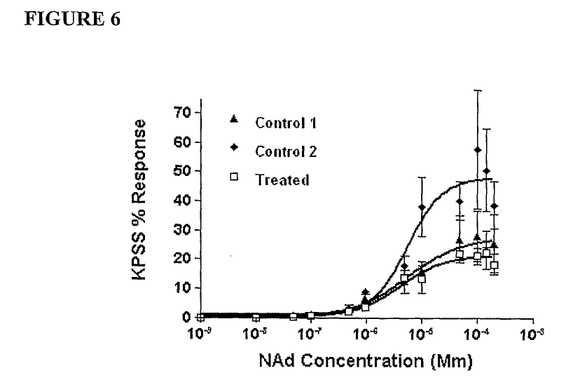

Isolated segments of vasa differentia were placed in organ baths to measure contraction of tissues in response to noradrenaline (Nad). Nad produced a dose-dependent contraction of the tissue. Exposure of animals to repeated restraint stress produced an increase in the sensitivity of smooth muscles to Nad. FIG. 6 shows the dose response curves for muscle contraction vs dose of noradrenaline constructed for isolated preparations from animals that were either not stressed (control 1), exposed to daily restraint for 14 days (control 2) and exposed to daily restraint for 14 days while housed in the presence of a composition containing 0.03% volume CH, 0.03% volume TH and 0.015% volume ap. There was an increase in sensitivity of smooth muscle to noradrenaline following repeated restraint stress. However, similar exposure to repeated restraint stress in animals housed in the presence of the odour did not result in increased sensitivity of the smooth muscle to Nad.

### Example 6

Optimisation of the amounts and ratios of CH, TH and ap were performed using different amounts of each component and different ratios of each component. Comparisons were made with a 0.3% lavender composition, which is commonly used in aromatherapy as a stress reliever. The level of sympathetic neurotransmission was used as a measure of stress using electrophysiological studies. The vas deferens from mice were used to study the sympathetic nervous system. Mice were either untreated, stressed without odour or stress with odour.

The electrophysiological studies have revealed that chronic stress exposure induces upregulation of sympathetic neurotransmission and increased responsiveness to exogenous adrenaline. However, a combination of CH, TH and ap was found to reduce stress at the sympathetic nervous system level.

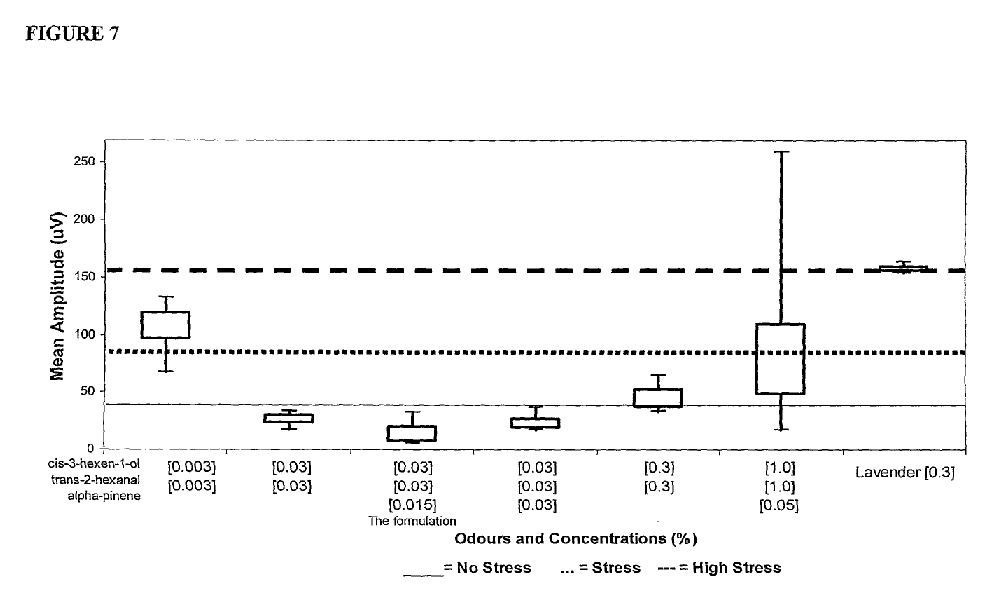

FIG. 7 demonstrates that a number of ratios of the combination of CH, TH and ap reduced stress as measured by sympathetic neurotransmitter release. The control, no exposure to the combination of the invention indicated high stress at a mean amplitude of 140 uV (black line). Interestingly 0.3% lavender increased the stress experienced by the animals.

As can be seen by FIG. 7, the combination of the invention was most effective at relieving stress at ratios of CH:TH:ap, 1:1:0.5 to 1:1:1 and in amounts of 0.03 to 0.3% for CH and TH and 0.015 to 0.15% for ap.

### Example 7

Safety data for the components of the combination of the invention have been obtained by applying the chemicals to smooth muscle in organ bath experiments. The mouse vas deferens was isolated and placed in an electrophysiological organ bath. Extracellular recording of transmitter release from sympathetic varicosities was recorded before and after both application of each of the chemicals CH, TH or ap. The effect (“success rate”) of each of the chemicals was evaluated over 40 minutes. Success rate was measured as (number of successful transmissions/number of nerve stimulations)×100. Following this measurement, the chemicals were washed out.

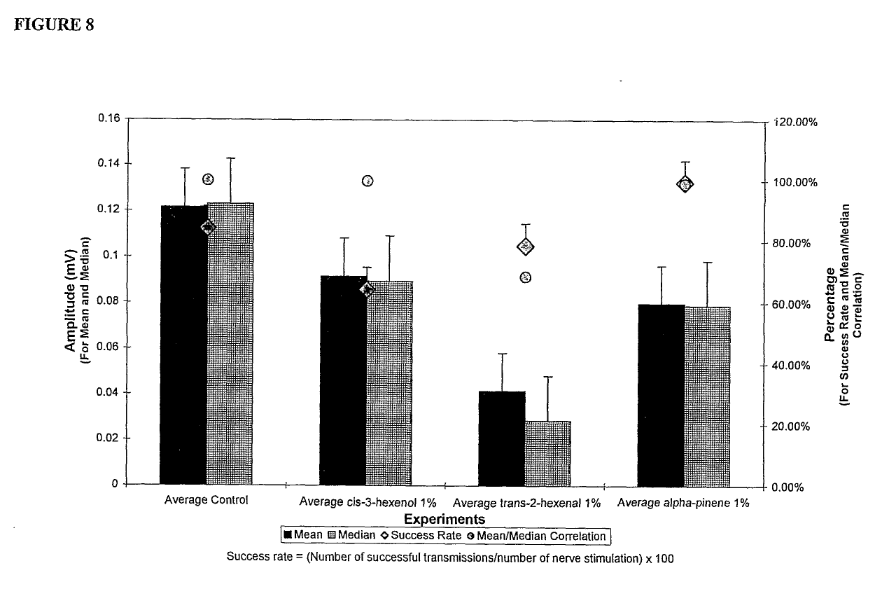

As can be seen in FIG. 8, the components of the combination of the invention, CH, TH and ap did not significantly affect the function of smooth muscle, indicating that even at concentrations well above those proposed for the present composition (1.0% vol), the components of the composition are safe. For TH there was some effect, at the high concentration used, on the smooth muscle. At the high concentrations, TH relaxed smooth muscle cells, providing a probable physiological effect of relaxing smooth muscle cells of the airway.

Exposing mice to 33 times the optimum concentration of the composition (CH:TH:ap 1%:1%:0.5%) for 42 days did not induce any noticeable adverse effects. However, it was observed that 92% of the animals would spend most of their time near the corner of the cage where the odour was applied. Normally experimental animals avoid unpleasant or harmful chemicals.

### Example 8

### Delay or Abolishment of the Development of Hypertension in Spontaneously Hypertensive Rats (SHR) by Exposure to the Composition of the Invention

Background. Numerous studies have implicated a centrally mediated increase in sympathetic nervous system NS activity and altered baroreflex function in the development and maintenance of hypertension. Primary hypertension in humans has also been shown to be associated with an increase in plasma levels of noradrenaline (Goldstein, 1981) and increased cardiac and renal noradrenaline spill over (Esler et al., 1988a,b). Microneurograph recordings of the peroneal nerve (Anderson et al., 1989; Grassi et al., 1998) and single unit recordings (Grassi et al., 1998) in hypertensive patients indicate increased sympathetic nerve discharge that is driven by the central nervous system.

The development of hypertension in SHRs involves an increase in sympathetic NS activity. This increase in sympathetic NS activity leads to a cascade of effects starting with vasoconstriction and increased cardiac output, thus a rise in blood pressure which then activates the renin-angiotensin system. The increased sympathetic NS activity also contributes to trophic effects on vascular smooth muscle and thus enhanced responses of smooth muscle to noradrenalin.

At birth the pups from SHR parents are normotensive but develop hypertension during the first four weeks postnatal. It is thought that exposure to stress during this postnatal period is responsible for the development of hypertension. This experiment demonstrates that concurrent exposure of laboratory animals to chronic stress and the combination of the invention prevents the increase in sympathetic nerve activity and increase in sensitivity of blood vessel muscle to the sympathetic neurotransmitter (noradrenaline).

Method. Spontaneously hypertensive rats about to give birth are exposed to either vehicle odour (control) or the composition of the invention (CH:TH:ap=0.03%:0.03%:0.015%) to inhibit the increase in sympathetic nerve activity following exposure to chronic stress. Following birth the pups continue to be exposed to the respective odour for up to 6 weeks. At two weeks postnatal the rats are implanted with telemetry transmitters and allowed to recover from the surgery over two weeks. Telemetry data is then recorded over two weeks. On the last week the odours are withdrawn while telemetry data are continually recorded. Also blood pressure measurements are taken throughout the treatments using a tail cuff. The development of hypertension is closely monitored and compared between treatment groups while monitoring the telemetry data such as heart rate, ECG, core body temperature and locomotor activity.

Results. The development of hypertension in spontaneously hypertensive rats (SHRs) is delayed by exposure to the composition of the invention.

### Example 9

### Odour-Induced Stress Relief

### Impact On Neural Excitability and Synaptic Plasticity Induced by Chronic Stress and Demonstration that High Doses of the Combination of the Invention do not Lead to Structural or Functional Changes to Neurons

Aims. (1) Demonstrate that exposure to chronic stress-relieving odours does not produce significant abnormalities in neuronal excitability or synaptic plasticity in brain areas processing olfactory information; and (2) determine whether the effects of chronic stress on neuronal excitability and synaptic plasticity in these areas are reduced or abolished by exposure to the combination of the invention.

Background. The reduction in chronic stress responses by specific odours or odour mixtures indicates that odour-evoked effects responsible for this specific reduction of autonomic responses to chronic stress are not likely to occur in midbrain and brainstem centres controlling all autonomic responses and output. Instead, odour effects are most likely in neural centres processing olfactory information upstream from centres generating autonomic responses.

After the first central sensory processing of olfactory receptor activation in the olfactory bulb, the lateral olfactory tract (LOT) conveys integrated olfactory information to important limbic centres of the amygdala and the hippocampus via the entorhinal cortex (Haberly, 2001; McNamara et al., 2004). The LOT also directly innervates the olfactory (piriform) cortex (McNamara et al., 2004), the output of which in turn also projects to the amydala and entorhinal cortex (Haberly, 2001). The hippocampus, amygdala and piriform cortex all have projections to centres generating autonomic responses (Haberly, 2001;Sah et al., 2003), and are thus possible sites for odour effects.

Of all sensory modes, the sense of smell (olfaction) has the most powerful impact on emotional and stress responses and is capable of forming and triggering strong emotional memories in animals and humans. The strong and lasting mental effects of odours suggests that olfactory sensations are capable of positively or negatively influencing long term synaptic plasticity as part of the neural response to odours. Several forms of long term synaptic plasticity have been demonstrated in many brain areas receiving olfactory inputs, particularly the piriform cortex, amygdala and hippocampus (Sevelinges et al., 2004;Mouly & Gervais, 2002;Sah et al., 2003; Lynch, 2004).

The piriform cortex is thought to be responsible for forming neural representations of odours or odour mixtures (Haberly, 2001). Piriform cortex has a simple three layered organisation, with large pyramidal neurones extending apical dendrites through layer I to the cortex surface from their somata in layers II/III (Haberly, 2001). LOT inputs synapse profusely on pyramidal cell dendrites and somata and exhibit NMDA receptor activation-dependent long term potentiation (LTP) (Kanter & Haberly, 1990;Haberly, 2001).

The entorhinal cortex/hippocampus is vital for distinguishing between different odours and for the formation and retention of memories for odours and other sensory inputs in rodents and man (Alvarez et al., 2002; Eichenbaum et al., 1983). LTP has been demonstrated for many synaptic connections in entorhinal cortex and hippocampus (Lynch, 2004), but this experiment focuses on NMDA receptor-dependent LTP in hippocampal CA1 neurones, as this form of synaptic plasticity has been shown to be differentially modulated by acute and chronic stress (Wolf, 2003; Lynch, 2004).

The amygdala is a key limbic structure for emotional and stress responses evoked by many different senses (Sah et al., 2003; Lynch, 2004). It has a complex multi-nuclear structure (Sah et al., 2003). Olfactory inputs have access to the amygdala at an earlier stage of processing than other sensory inputs, which are typically processes and integrated by the thalamus first. LOT and piriform cortex projections are selectively routed through the cortical and medial nuclei of the amygdala (McNamara et al., 2004), in contrast to most other sensory inputs from cortical areas or the thalamus, which enter through the lateral or basolateral nuclei (Sah et al., 2003). While neurones of the basolateral and lateral nuclei show LTP associated with classical conditioning to stressful stimuli (Sah et al., 2003), relatively little is known about synaptic plasticity in the cortical and medial nuclei. Recently, fear conditioning using an odour as the conditioned stimulus was shown to produce a long-lasting potentiation of LOT-evoked extracellular field potentials in the cortical nucleus (Sevelinges et al., 2004).

Methods. Extracellular field potentials (EFPs) or whole cell (WC) patch clamp recordings from individual neurones are made in slices of brain tissue from rats aged 2-4 weeks and maintained in vitro for 6-8 hours after preparation (Baraban et al., 1997; Bellingham & Berger, 1996). Adult stress responses to restraint develop by 10 days post birth in rats (Sullivan, 2005). Animals are deeply anaesthetised with sodium pentobarbitone (100 mg/kg i.p.), then decapitated and the forebrain rapidly removed and cooled in ice-cold ACSF (composition (in mM): 130 NaCl, 26 NaHCO₃, 10 Glucose, 3 KCl, 1.25 NaPO₄, 1 MgCl₂, 2 CaCl₂, gassed with 95% O₂-5% CO₂, pH 7.4). After mounting on a microtome platform, 200-400 μm thick transverse sections of relevant areas of the forebrain is cut in ice-cold ACSF, and then held in ACSF at room temperature until recording.

Synaptic inputs are evoked by electrical stimulation of the LOT (in piriform cortex or amygdala) or Schaffer collaterals (in hippocampus). EFPs evoked by these inputs are recorded from the CA1 region of the hippocampus, layers II/III of the pyriform cortex or the central/medial nuclei of the amygdala in slices placed in an interface chamber. After recording a stable baseline period of >10 min, LTP is induced by a single high frequency burst stimulation (100 Hz for 1 min) or theta burst stimulation (10 bursts of four 100 Hz pulses, with each burst separated by 200 ms) (Lynch, 2004). Changes of the EFP slope and amplitude are monitored for >30 min after LTP induction and compared to baseline measurements to assess LTP.

WC recordings are made from visually identified neurones in the same areas used for EFP recording in slices placed in a superfused chamber on the microscope stage (Baraban et al., 1997; Bellingham & Berger, 1996). Neuronal excitability are assessed by injection of current steps to evoke repetitive action potential firing and measure input resistance. Pharmacologically isolated excitatory or inhibitory synaptic inputs evoked by electrical stimulation are recorded, and their response to paired pulse stimulation (short term plasticity) or LTP induction stimuli assessed.

Statistical comparisons (unpaired 2 tail t test or ANOVA) of measured mean responses are made between groups of (1) rats exposed to stress-relieving odours at a range of concentrations and non-exposed littermates, and between (2) rats exposed to chronic stress conditions alone or with concurrent exposure to stress-relieving odours.

Results: Chronic administration of the combination of the invention prevents the alterations in synaptic structure and function of hippocampal neurons. This demonstrates that stress-relieving odours are not harmful to normal brain function and that these odours (at the concentration demonstrated to have an effect) reduce or prevent known effects of chronic stress on central neurones.

### Example 10

### The Combination of the Invention Moderates the Physiological Effects of Chronic Stress by Reducing the Levels of Adrenocorticotropic Hormone (ACTH), Cortisol and Catecholamines

Aims. (1) To measure (in mice) the levels of key indicators of stress such as adrenocorticotrophic hormone (ACTH), corticosterone and catecholamines from blood samples of non-stressed, restraint stressed in the presence of vehicle (tri-ethyl citrate) odour and restraint stressed in the presence of the combination of the invention. The results are then correlated with electrophysiological data obtained from studying neurotransmission from the same animals using both electrophysiological analysis of transmitter release and the responses of smooth muscle to Nad. (2) To measure (in sheep) the levels of key indicators of stress such as adrenocorticotrophic hormone (ACTH), cortisol and catecholamines from blood samples of non-stressed, restrain stressed in the presence of vehicle (tri-ethyl citrate) odour and restrain stressed in the presence of the combination of the invention. In these animals the level of amylase in saliva is also measured and correlated with the levels of adrenocorticotrophic hormone (ACTH), cortisol and catecholamines from blood samples.

Background. The response to stress has been shown to be affected by olfaction (reviewed Kelly et al., 1997). For example, bilateral olfactory bulbectomised rats show extremely high levels of glucocorticoids in the presence of stressors (O'Connor & Leonard, 1984). Olfactory bulbectomised rats show hyperactivity in their hypothalamo-pituitary-adrenocortical axis during stress, with higher plasma levels of adrenocorticotropic hormone and corticosterone after 15 minutes of immobilising stress (Marcilhac et al., 1999). Odours from various sources are known to affect animals; for example, rat odour has been shown to increase sympathetic neurotransmission by 318% in mice (D'Arbe et al., 2002). Although there are many more anecdotal and speculated effects of odours on animal and human behaviour, it is often extremely difficult to establish a controlled environment and control subjects to determine if these physiological effects are significant. Methods. Using telemetry transmitters heart rate, ECG, core body temperature and locomotor activity were continually monitored in the whole animal, (Einstein et al., 2000; Einstein et al., 2004) and a bioassay kit was used to measure levels of ACTH, cortisol and catecholamine. Such assay kits are readily available from a number of laboratory supply stockists. The activity of the sympathetic nervous system was assessed using electrophysiological methods (Lavidis and Bennett, 1992, 1993a, 1993b; D'Arbe et al., 1999; D'Arbe et al., 2002). In all experiments, statistical comparisons (unpaired 2 tail t test or ANOVA as appropriate) of measured mean responses are made between groups.

Experiment 1. Mice were repeatedly exposed to restraint stress while exposed to either vehicle or the combination of the invention (CH:TH:ap=0.03%:0.03%:0.015%) for 14 days. The animals were exposed to the formulation or vehicle by addition of 0.2 mL, 2 times per day to their bedding at the bottom of the cage. After 14 days the mice were killed by decapitation. Blood is collected to analyse the levels of ACTH, corticosterone and catecholamines. The timing of the collection of blood is carefully considered to avoid large variations of these hormones and transmitters by diurnal rhythm.

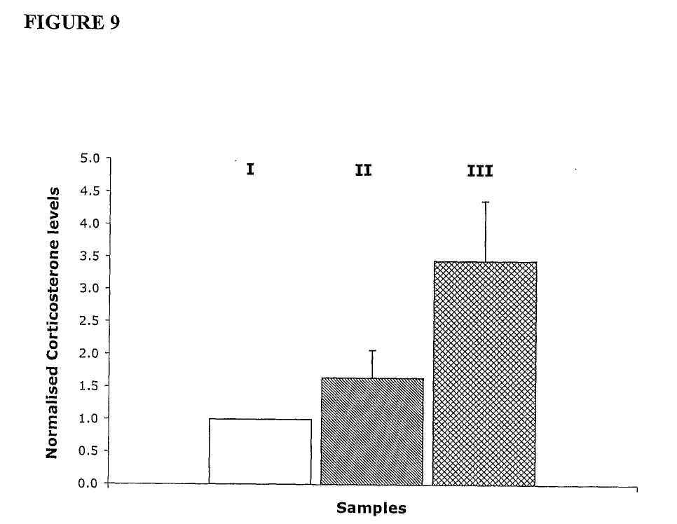

Results. The results demonstrated that stress induces an increase in the levels of ACTH, corticosterone and catecholamines but in the presence of the combination of the invention there is a significant attenuation to the stress response and these results correlate with previous electrophysiological and telemetry data. The results of the corticosterone levels are given in FIG. 9 and show that the formulation reduced stress in the mice.

Experiment 2. Sheep are housed in the absence or presence of odours and exposed to intermittent (1 hour per day) restraint stress for 7 days. One day before the restraint stress (control) and on days 1, 3, 5 and 7 of the restraint stress, blood is collected from the animal (1 ml, from a catheter in the jugular vein) every 15 minutes during the exposure to stress and for the following hour. Levels of ACTH, cortisol and catecholamines in the blood are measured. The timing of the collection of blood is carefully considered to avoid large variations of these hormones and transmitters by diurnal rhythm.

Results. The results demonstrated that stress induces an increase in the levels of ACTH, cortisol and catecholamines but in the presence of the combination of the present invention (CH:TH:ap=0.03%:0.03%:0.015%) there is a significant attenuation of the stress response.

Experiment 3. A sample of saliva is taken with a test stick containing an amylase sensing chip from sheep exposed to different conditions (unstressed, stressed exposed to vehicle odour and stressed exposed to the combination of the invention (CH:TH:ap=0.03%:0.03%:0.015%). The amylase levels are assayed using a commercial test kit. The stressor used is restraint for one hour in a holding pen.

Results. The combination of the invention is effective in reducing the level of amylase in saliva following chronic exposure to stress in sheep.

### Example 11

### Effect of the Formulation on Humans

Background. Kirschbaum et al., (1993) designed the Trier Social Stress Test (TSST), which involves a 10 minute anticipation period, in which the subject is told to prepare for a public speaking task (i.e., presenting one's personal strengths and qualifications for a job) followed by a 10-minute session, during which the subject actually delivers the speech to an unfamiliar and non-responsive, 3-person panel of experts, followed by a mental arithmetic task performed out loud (i.e., “subtract 13 from 2034 as quickly and accurately as possible . . . ”). When a subject makes a mistake they are told to stop and to start from the beginning. This procedure, often used in psychoneuroendocrinological research, has shown to induce significant physiological and psychological responses to stress in both healthy subjects and stressed participants (Domes et al., 2002).

Modified versions of the TSST have been created. Gold et al.'s procedure (2002) consists of a 5-minute speech task, in which participants were asked to imagine they had been caught for a traffic violation (e.g., running a red light) and had to defend themselves at the traffic court. They were allowed two minutes for preparation and three minutes to deliver the speech. Speeches were delivered in front of a video camera, and subjects were told that their performance would later be rated by experts for content and style. This was followed by a 5-minute mental arithmetic task with harassment, during which participants were asked to add numbers out loud at the pace set by an audiotape.

Method. A 4×2×2 mixed study design tested the effect of the composition on stress. The study used four groups (non/low-stressed subjects, high-stressed subjects, mildly depressed, and moderately/severely depressed subjects), and two levels within groups (stress-induced testing, without-stress testing). The subjects were recruited by advertisement and referral by clinicians as appropriate to their classification as normal or depressed. Stress was measured via salivary cortisol assays. Cortisol is analysed using (Elisa) Cortisol RIA kit (no. DSL-2000) from Diagnostic Systems Ltd., Parramatta, NSW, Australia. Stress was induced by TSST. Half of the subjects in each block received the formulation during stress induction. The composition was a mixture of CH, TH and ap diluted with triethyl citrate (TEC) at a ratio of CH:TH:ap of 0.03%:0.03%:0.015%. Controls received the vehicle solution only, 100% of triethyl citrate, which has paraffin-like smell with no known mental effects. Using a pipette, 0.5 ml of the formulation was put on a sheet of absorbent paper (5×10 mm) and attached to a collar chain about 10 cm below the subject's nose during stress induction. Subjects were instructed to breathe normally through their nose throughout the task.

Results: The formulation is effective in reducing the level of cortisol in saliva following exposure to stress in normal, stressed and depressed humans.

### Example 12

### a) Effects of the Formulation on Formation, Retention and Extinction of Fear-Related Behaviours

The amygdala is an important processing centre for the learning and retention of a range of fear-related mental disorders, such as fear conditioning, phobias and anxieties such as post-traumatic stress disorders. Most sensory inputs enter the amydala via the basolateral nuclei complex, which is the site of long-term synaptic plasticity thought to underlie the acquisition and retention of fear conditioning. Output from the basolateral nuclei is to the central nucleus, which then projects to all major neural control centres for autonomic and neurohumoral stress responses. However, the olfactory system has unique connections to the amygdala. The olfactory bulb is directly and densely connected to the corticomedial nuclei of the amygdala, with few inputs to the basolateral nuclei. This separate processing pathway for olfactory information in the amygdala suggests that olfactory memory may be due to synaptic plasticity at a different site in the amygdala complex, and that olfactory memory may be capable of positively or negatively modulating fear conditioning using other sensory modalities. Fear conditioning using odorants as a conditioned (learned stimulus) produces robust emotional and physiological fear responses in adult animals. Odorants are also reported to be powerful triggers for fear-related disorders, such as post-traumatic stress disorders. Evidence suggests that these disorders are associated with abnormalities of normal stress responses, with high or prolonged sympathetic activation being part of the inappropriate response.

This experiment establishes that the composition prevents or reduces acute and chronic stresses which could establish post-traumatic stress disorders in people in occupations or situations associated with higher likelihood of developing this disorder (e.g. persons in civil or medical emergency, police, military, prison). Exposure of subjects to a composition containing a ratio of CH:TH:ap of 0.03%:0.03%:0.015% for 15 minutes following establishment of fear conditioning more rapidly leads to extinction of learned fear responses. This establishes that the composition is a suitable adjuncts to conventional psychotherapy/psychiatric treatments for these disorders.

### b) Effects of the Formulation on Formation, Retention and Extinction of Conscious Learning

The hippocampus is an important brain centre for various forms of conscious learning. The entorhinal cortex/hippocampus is vital for distinguishing between different odours and for the formation and retention of memories for odours and other sensory inputs in rodents and humans. Long term changes in synaptic transmission has been demonstrated for many synaptic connections in entorhinal cortex and hippocampus. Chronic stress is associated with poorer formation and retention of memories and is also associated with changes in hippocampal structure, including reduced neuron numbers, lower synaptic densities, impaired neurogenesis from adult neural stem cells

This experiment establishes the composition having a ratio of CH:TH:ap of 0.03% 0.03%:0.015% is a suitable agent for prevention or reduction of the effects of chronic stress on learning and memory. Exposure of a subject to the composition for up to 15 minutes before and during formation of memories enhances formation and retention of memories under normal conditions. This experiment also establishes the formulation as a suitable agent for enhancing learning and memory under conditions in which this was desirable.

### Effects of the Formulation on Depression

Depression is associated with hyper-responsiveness of the brain stress control system, due to genetic factors or due to aversive stimuli that may occur during early development or adult life. The locus of psychobiological changes underlying depression remains unknown. Pharmacological treatment of depression with anti-depressants can reverse effects of chronic stress on brain regions such as the hippocampus. These experiments demonstrate that exposure to the formulation having a ratio of CH:TH:ap of 0.03% 0.03%:0.015% when depression is present can reduce or abolish physiological, anatomical and behavioural manifestations of depression in animals models of depression or humans. The effects of the formulation were assessed using the animal model of learned helplessness, in which rats are exposed to inescapable and uncontrollable shocks then subsequently fail to flee from the shocks even when an escape route is available, with the percentage of rats showing helpless behaviour as the measured outcome. Other models of depressions may also be used, such as behaviour tests, for example, forced swim test or tail suspension test, in which animals are exposed to continued stress (swimming, hanging by their tails) until movements to escape from the test situation cease, with the length of time until escape movements stop being the measured outcome or chronic stress models, in which animals are exposed to relatively moderate stressors (wet bedding, constant lighting or food deprivation), with neurochemical, neuroendocrine and neuroimmune responses and animal coat condition being the common measures of outcome. In humans, depression can be assessed by standard clinical instruments, including but not limited to the Diagnostic and Statistical Manual (DSM-IV) of the American Pyschiatric Association, the International Classification of Diseases (ICD-10) of the World Health Organisation, and rating instruments such as the Hamilton Depression Scale. This experiment establishes the formulation as a suitable agent for treatment of depression alone, or as an adjunct with pharmacological or psychotherapeutic treatments.

### Example 13

### Morphological Effects of the Formulation on the Brain

### Chronically Administered Formulation Prevents the Alterations in Synaptic Structure and Function of Olfactory-Limbic Neural Centres Induced by Chronic Stress.

Background. Design-based neuro-stereological methods quantify the numbers neurons, cell soma size, and numbers of input synapses of the specific limbic regions that receive integrated olfactory information, such the amygdala, hippocampus, entrorhinal cortex, and piriform (olfactory) cortex (Gundersen et al 1999; West 1999).

Method. Animals were deeply anaesthetised with sodium pentobarbitone (100 mg/kg i.p.), and perfused with formalin. Limbic regions were embedded in resin and/or wax and were serially sectioned at 80 μm through the entire structure of interest (e.g. the relevant limbic centre). Gross volume of the relevant limbic structures were determined by the Cavalieri-estimator method. Numbers of neurons were counted using the optical fractionator method (i.e. selected sections at a set interval with the first section chosen at random; uniform random sampling through the entire structure of interest). Synaptic number detection were performed using the physical dissector method from resin sections at the electron microscopic level. Size of neurons was determined using the nucleator and planar rotator methods. Statistical comparisons (unpaired 2 tail t test or ANOVA as appropriate) of measured mean responses were made between groups of 1) rats exposed to stress-relieving odours at a range of concentrations and non-exposed littermates, and between 2) rats exposed to chronic stress conditions alone or with concurrent exposure to stress-relieving odours. This documents possible changes in synaptic structure, neuronal number and size for a given limbic region. Any changes in neuronal excitability were correlated with neuronal morphology and number.

Results. Chronic administration of the formulation prevents alterations in synaptic plasticity specific limbic regions that receive integrated olfactory information, such the amygdala, hippocampus, entorhinal cortex, and piriform (olfactory) cortex and high doses of the formulation does not lead to structural changes to the neurons of these limbic regions.

### REFERENCES

Akutsu H, Kikusui T, Takeuchi Y, Sano K, Hatanaka A, Mori Y (2002) Alleviating effects of plant-derived fragrances on stress-induced hyperthermia in rats. Physiol Behav 75:355-360.
Alvarez P, Wendelken L, Eichenbaum H (2002) Hippocampal formation lesions impair performance in an odor-odor association task independently of spatial context. Neurobiol Learn Mem 78:470-476.
Anderson E A, Sinkey C A, Lawton W J, Mark A L (1989) Elevated sympathetic nerve activity in borderline hypertensive humans. Evidence from direct intraneural recordings. Hypertension 14:177-183.
Baraban S C, Bellingham M C, Berger A J, Schwartzkroin P A (1997) Osmolarity modulates K+ channel function on rat hippocampal interneurons but not CA1 pyramidal neurons. J Physiol 498 (Pt 3):679-689.
Bellingham M C, Berger A J (1996) Presynaptic depression of excitatory synaptic inputs to rat hypoglossal motoneurons by muscarinic M2 receptors. J Neurophysiol 76:3758-3770.
Cacioppo J T (1994) Social neuroscience: autonomic, neuroendocrine, and immune responses to stress. Psychophysiology 31:113-128.
Cacioppo J T, Berntson G G, Malarkey W B, Kiecolt Glaser J K, Sheridan J F, Poehlmami K M, Burleson M H, Ernst J M, Hawkley L C, Glaser R (1998) Autonomic, neuroendocrine, and immune responses to psychological stress: the reactivity hypothesis. Annals of the New York Academy of Sciences 840:664-673.
Chrousos G P, Gold P W (1992) The concepts of stress and stress system disorders. Overview of physical and behavioral homeostasis. Journal of the American Medical Association 267:1244-1252.
Chrousos G P, McCarty R, Pacak K, Cizza G, Sternberg E, Gold P W, Kvetnansky R, eds (1995) Stress: Basic mechanisms and clinical implications, 1 Edition. New York: New York Academy of Sciences.
D'Arbe M, Chin I, Einstein R, Lavidis N A (1999) Stress induced changes in transmitter release from sympathetic varicosities of the mouse vas deferens. J Auton Nerv Syst 76:146-152.
D'Arbe M, Einstein R, Lavidis N A (2002) Stressful animal housing conditions and their potential effect on sympathetic neurotransmission in mice. Am J Physiol Regul Integr Comp Physiol 282:R1422-1428.
Domes G, Heinrichs M, Reichwald U, Hautzinger M (2002) Hypothalamic-pituitary-adrenal axis reactivity to psychological stress and memory in middle-aged women: High responders exhibit enhanced declarative memory performance. Psychoneuroendocrinology, 27:843-853.
Dunn J D, Arimura A, Scheving L E (1972) Effect of stress on circadian periodicity in serum LH and prolactin concentration. Endocrinology 90:29-33.
Eichenbaum H, Morton T H, Potter H, Corkin S (1983) Selective olfactory deficits in case H.M. Brain 106 (Pt 2):459-472.
Einstein R, Rowan C, Billing R, Lavidis N A (2000) The use of telemetry to refine experimental technique. In: Progress in the reduction, refinement and replacement of animal experimentation (Balls M, Van Zeller A M, Halder M E, eds), pp 1187-1197. Amsterdam: Elsevier Science.
Einstein R, Billing R, Singh A, Chin 1 (2004) Implanted telemetry transmitters alter the nordrengeric response in vas deferens from mice. Alternatives to Laboratory Animals 32:171-176.
Esler M, Jennings G, Korner P, Willett I, Dudley F, Hasking G, Anderson W, Lambert G (1988a) Assessment of human sympathetic nervous system activity from measurements of norepinephrine turnover. Hypertension 11:3-20.
Esler M D, Jennings G L, Lambert G W (1988b) Release of noradrenaline into the cerebrovascular circulation in patients with primary hypertension. J Hypertens Suppl 6:S494-496.
Fraser D, Ritchie J S, Fraser A F (1975) The term “stress” in a veterinary context. British Veternary Journal 131:653-662.
Gamallo A, Alario P, Villanua M A, Nava M P (1988) Effect of chronic stress on the blood pressure in the rat: ACTH administration. Hormone and Metabolic Research 20:336-338.
Gold P W, Chrousos G P (2002) Organization of the stress system and its dysregulation in melancholic and atypical depression: high vs low CRH/NE states. Mol Psychiatry 7:254-275.
Goldstein D S (1981) Plasma norepinephrine in essential hypertension. A study of the studies. Hypertension 3:48-52.
Grassi G, Cattaneo B M, Seravalle G, Lanfranchi A, Mancia G (1998) Baroreflex control of sympathetic nerve activity in essential and secondary hypertension. Hypertension 31:68-72.
Gundersen H J G, Jensen E B V, Kieu K, Nielsen J (1999) The efficiency of systematic sampling in stereology—reconsidered. J. Microscopy 193:199-211.
Haberly L B (2001) Parallel-distributed processing in olfactory cortex: new insights from morphological and physiological analysis of neuronal circuitry. Chem Senses 26:551-576.
Kanter E D, Haberly L B (1990) NMDA-dependent induction of long-term potentiation in afferent and association fiber systems of piriform cortex in vitro. Brain Res 525:175-179.
Kelly J P, Wrynn A S, Leonard B E (1997) The olfactory bulbectomized rat as a model of depression: an update. Pharmacol Ther 74:299-316.
Kirschbaum C, Pirke K-M, Hellhammer D H (1993). The Trier Social Stress Test—A tool for investigating psychobiological stress responses in a laboratory setting. Neuropsychobiology, 28, 76-81.
Lavidis N A, Bennett M R (1992) Probabilistic secretion of quanta from visualized sympathetic nerve varicosities in mouse vas deferens. J Physiol 454:9-26.
Lavidis N A, Bennett M R (1993a) Probabilistic secretion of quanta from successive sets of visualized varicosities along single sympathetic nerve terminals. J Auton Nerv Syst 43:41-50.
Lavidis N A, Bennett M R (1993b) Sympathetic innervation of the surface of the mouse vas deferens. J Auton Nerv Syst 45:87-100.
Lynch M A (2004) Long-term potentiation and memory. Physiol Rev 84:87-136.
Marcilhac A, Faudon M, Anglade G, Hery F, Siaud P (1999) An investigation of serotonergic involvement in the regulation of ACTH and corticosterone in the olfactory bulbectomized rat. Pharmacol Biochem Behav 63:599-605.
McCarty R, Kvetnansky R, Kopin I J (1981) Plasma catecholamines in rats: daily variations in basal levels and increments in response to stress. Physiology and Behavior 26:27-31.
McNamara A M, Cleland T A, Linster C (2004) Characterization of the synaptic properties of olfactory bulb projections. Chem Senses 29:225-233.
Mouly A M, Gervais R (2002) Polysynaptic potentiation at different levels of rat olfactory pathways following learning. Learn Mem 9:66-75.
O'Connor W T, Leonard B E (1984) Effect of stress on the secretion of corticosterone in the bilbectomized rat model of depression. Irish J Med Sci: 153-226.
Sah P, Faber E S, Lopez De Armentia M, Power J (2003) The amygdaloid complex: anatomy and physiology. Physiol Rev 83:803-834.
Sano A, Sei H, Seno H, Morita Y, Moritoki H (1998) Influence of cedar essence on spontaneous activity and sleep of rats and human daytime nap. Psychiatry Clin Neurosci 52:133-135.
Sano K, Tsuda Y, Sugano H, Aou S, Hatanaka A (2002) Concentration effects of green odor on event-related potential (P300) and pleasantness. Chem Senses 27:225-230.
Sevelinges Y, Gervais R, Messaoudi B, Granjon L, Mouly A M (2004) Olfactory fear conditioning induces field potential potentiation in rat olfactory cortex and amygdala. Learn Mem 11:761-769.
Singh A, Einstein R, Lavidis N (2001) Effects of restraint stress on responsiveness of atria and vas deferens in Sprague-Dawley rats. J Auton Pharmacol 21:255-261.
Sullivan R M (2005) Developmental changes in olfactory behavior and limbic circuitry. Chem Senses 30 Suppl 1:i152-i153.
West M J (1999) Stereological methods for estimating the total number of neurons and synapses: issues of precision and bias. Trends in Neurosci. 22:51-61.
West M J (2002) Design-based sterological methods for counting neurons. Prog. Brain Res. 135:43-51.
Wolf O T (2003) HPA axis and memory. Best Pract Res Clin Endocrinol Metab 17:287-299.

1. A composition comprising cis-3-hexen-1-ol, trans-2-hexenal and α-pinene.

2. The composition according to claim 1, wherein the cis-3-hexen-1-ol is present in an amount of 0.003% to 3% by volume, the trans-2-hexenal is present in an amount of 0.003% to 3% by volume and the α-pinene is present in an amount of 0.001% to 0.15% by volume,

3. The composition according to claim 1, wherein the ratio of cis-3-hexen-1-ol:trans-2-hexenal:α-pinene is in the range of 0.5-2:0.5-2:0.25-2.

4. The composition according to claim 3, wherein the ratio of cis-3-hexen-1-ol:trans-2-hexenal:α-pinene is about 1:1:0.505.

5. The composition according to claim 1 further comprising at least one of a carrier, an adjuvant, an excipient, a further odor component, a cleaning component, a coloring or polishing component, an adhesive or a coating component.

6. The composition according to claim 1, wherein the composition is a cleaning composition, a personal hygiene composition, a massage oil, a laundry detergent, a furniture polish, a shoe polish, a cosmetic product, an air freshener, a fragrance composition, an ink composition, an adhesive composition or a coating composition.

7. An article of manufacture comprising or impregnated with a combination comprising cis-3-hexen-1-ol, trans-2-hexenal and α-pinene.

8. The article of manufacture according to claim 7, wherein the ratio of cis-3-hexen-1-ol:trans-2-hexenal:α-pinene is in the range of 0.5-2:0.5-2:0.25-2.

9. The article of manufacture according to claim 8, wherein the ratio of cis-3-hexen-1-ol:trans-2-hexenal:α-pinene is about 1:1:0.5.

10. The article of manufacture according to claim 7, wherein the article is paper, cardboard, plastic or fabric.

11. The article of manufacture according to claim 10, wherein the article is impregnated paper or cardboard for use in freshening or perfuming a home, office or car, cardboard or plastic strips for use in a battery operated odor emanating device or an odor emanating device attached to a power point, writing paper, an envelope, a book, a newspaper, an instruction manual, a magazine, a clean cloth, a duster, a shoe polishing cloth, a handkerchief, bedroom, bathroom or table linen or plastic or polymeric items able to release odor.

12. The article of manufacture according to claim 7, wherein the combination is adapted for slow release delivery.

13. A method of relieving or preventing the effects of chronic exposure to stress in a subject comprising exposing the subject to a combination or composition comprising cis-3-hexen-1-ol, trans-2-hexenal and α-pinene.

14. The method according to claim 13, wherein the cis-3-hexen-1-ol is present in an amount of 0.003% to 3% by volume, the trans-2-hexenal is present in an amount of 0.003% to 3% by volume and the α-pinene is present in an amount of 0.001% to 0.15% by volume of the combination or composition.

15. The method according to claim 13, wherein the ratio of cis-3-hexen-1-ol:trans-2-hexenal:α-pinene is in the range of 0.5-2:0.5-2:0.25-2.

16. The method according to claim 15, wherein the ratio of cis-3-hexen-1-ol:trans-2-hexenal:α-pinene is about 1:1:0.5.

17. The method according to claim 13, wherein the composition or combination is adapted for slow release delivery.

18. The method according to claim 13, wherein the subject is a human, a domestic or agricultural animal, an animal kept in captivity or a laboratory animal.

19. The method according to claim 13, wherein relieving or preventing the effects of chronic exposure to stress is relieving, preventing or delaying the onset of conditions selected from the group consisting of hypertension, immune suppression, depression, anxiety, loss of libido, indigestion, head aches, loss of memory, lack of concentration and learning difficulties.

20. The method according to claim 13, wherein memory, concentration and learning is improved in chronically stressed subjects.

21. The method according to claim 13, wherein the onset of post-traumatic stress syndrome is delayed or its severity reduced.

22. The method according to claim 13 further comprising another therapy for treating an adverse effect of chronic exposure to stress.

23. A method of relieving or preventing the effects of chronic exposure to stress in a subject comprising exposing the subject to a combination or composition comprising cis-3-hexen-1-ol and trans-2-hexenal.

24. (canceled)
